(19) United States
(12) Patent Application Publication
(10) Pub. No.: US 2013/0167602 A1
(43) Pub. Date: Jul. 4, 2013

(54) SYSTEMS AND METHODS FOR MAKING FOOD-BASED COMPOST

(71) Applicant: Daniel Blake, Alpine, UT (US)

(72) Inventor: Daniel Blake, Alpine, UT (US)

(73) Assignee: ECO SCRAPS, INC., Provo, UT (US)

(21) Appl. No.: 13/341,631

(22) Filed: Dec. 30, 2011

(51) Int. Cl. C05F 11/08; C12M 1/02

(52) U.S. Cl. 71/9; 435/290.2

(57) ABSTRACT

A method of making food-based compost includes mulching food to a predetermined maximum size, mixing mulched food with a filler to create a compost pre-mixture, periodically aerating the compost pre-mixture, and waiting a composting period until compost is formed from the compost pre-mixture. The compost does not include animal-based products.

### TECHNICAL FIELD

The present application related generally to composting systems and methods, and more particularly relates to systems and methods for making food-based compost.

### BACKGROUND

Traditional compost products include manures and other animal products, and green waste such as grass clippings, leaves, yard waste, etc. These types of compost are low in nutrients and provide limited value to a gardner by merely adding dead humic matter to the soil for consumption by bacteria. The compost is often mixed with dirt or other filler material that further reduces the concentration of nutrients. Further, the process of making this type of compost is very time intensive—usually taking in the range of at least 4-6 months.

Opportunities exist for improving the nutrient content and processing of compost.

### SUMMARY

One aspect of the present disclosure relates to a method of making food-based compost. The method includes mulching food to a predetermined maximum size, mixing mulched food with a filler to create a compost pre-mixture, aerating the compost pre-mixture, and waiting a composting period until compost is formed from the compost pre-mixture. The compost does not include animal-based products such as manure.

The method may include screening the mulched food to a maximum size of 1 inch. The method may include providing the filler at a size of no greater than ⅜ inch. The food may include fruits and vegetables. The fruits and vegetables may be provided at a ratio of 50 percent fruits and 50 percent vegetables by weight. The food may include citrus fruit at a ratio of no more than 30 percent by weight. A moisture content of the compost pre-mixture may be no more than 30 percent. A carbon to nitrogen ratio for the compost pre-mixture may be in the range of about 28:1 to 39:1. The filler may include sawdust. The compost pre-mixture may include a ratio of 1000 lbs. of mulched food for every ⅔ cubic yard of filler. The composting period may be not more than about 3 weeks. The aerating may occur every 2 to 4 days. The compost may not include dirt. The compost may not include animal products.

Another aspect of the present disclosure relates to a method of making food-based compost that includes mulching a plurality of fruits and vegetables, draining the mulched fruits and vegetables, and mixing the mulched fruits and vegetables with sawdust to create a compost pre-mixture having a carbon to nitrogen ratio in the range of about 28:1 to about 39:1. The method also includes elevating a temperature of the compost pre-mixture, periodically aerating the compost pre-mixture, and waiting a predetermined time until a compost is formed from the compost pre-mixture. The compost does not include animal-based products.

The method may include providing the plurality of fruits and vegetables at a ratio of 50/50 by weight. The method may include screening the mulched fruits and vegetables to a size of no greater than 1 inch. The method may include providing the compost pre-mixture at a moisture content of no greater than 30 percent. The aerating may occur at intervals of every 3 days or less. The predetermined time period may be no longer than about 3 weeks. The method may include piling the compost pre-mixture in windrows. The mulched fruits and vegetables may make up more than ½ of the compost pre-mixture by weight. The compost may not include dirt.

A further aspect of the present disclosure relates to a system for creating food-based compost. The system includes a food mulcher, a screener, a drainer, a food and filler mixing chamber, and an aerator. The food mulcher is configured to mulch food-based product. The screener is configured to remove mulched food-based product greater than a predetermined size. The drainer is configured to drain liquid from the mulched food-based product. The food and filler mixing chamber is configured to mix the mulched food-based product with a filler to form a compost pre-mixture. The aerator is configured to periodically aerate the compost pre-mixture until the compost pre-mixture changes to compost.

The food-based product may include a mixture of fruits and vegetables. The filler may include sawdust. The aerator may be a snow blower.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

The accompanying drawings illustrate a number of exemplary embodiments and are a part of the specification. Together with the following description, these drawings demonstrate and explain various principles of the instant disclosure.

FIG. 1 is a block diagram showing an example system in accordance with the present disclosure.

FIG. 2 is a block diagram showing another example system in accordance with the present disclosure.

FIG. 3 is a block diagram showing another example system in accordance with the present disclosure.

FIG. 4 is a flow diagram showing an example method of making compost in accordance with the present disclosure.

FIG. 5 is a flow diagram showing another example method of making compost in accordance with the present disclosure.

FIG. 6 is a block diagram showing an example system and method in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

The present disclosure relates to systems and method of creating compost using food waste as a primary component of the compost. Food waste is typically defined as product that is edible by humans but is no longer needed or suitable for its intended purpose as food. The food waste typically used for creating compost according to the present disclosure is fruit and vegetable food waste. Some embodiments in accordance with the present disclosure involve the use of food waste that does not include animal-based product such as meat, bone and manure. Some embodiments include food waste that comes from animals such as, for example, dairy based products, but does not include other animal-based products such as meat, bone or manure.

Another primary component of the compost in accordance with the present disclosure is a filler such as sawdust, sawdust shavings, and other wood-based product. The amount of filler added to the food waste may be altered to control, for example, moisture content, carbon to nitrogen ratio, and nutrients concentration for the compost.

A number of challenges exist when using food waste as a primary component of compost. Many types of composts use manures and green waste (e.g., grass clippings, leaves, yard waste, and other organic material that is typically not edible). These types of composts are relatively low in nutrient value and require an extended time period for completion of the composting process (e.g., at least 4 to 6 months). This extended time period results in larger space requirements in order to store the composting material, and also requires additional expense related to handling (e.g., aerating) the compost over that extended time.

Using food waste as a primary component of the compost in place of manure and green waste may significantly increase the nutrient content of the resulting compost and reduce the amount of time required for completing the composting process. However, the use of food waste may produce undesirable odors and may attract rodents and flies if not handled properly. The way in which the food waste is collected, prepared, aerated and mixed with a filler in accordance with the present disclosure reduces undesirable odors and provides heating of the compost material at a rate that avoids the attraction of flies and rodents. Further, the methods and systems of the present disclosure may significantly increase the speed of completing the composting process (e.g., in the range of less than a month rather than multiple months).

One example process for creating food-based compost in accordance with the present disclosure involves use of fruit and vegetable waste that is mulched to a size of no greater than 1 inch in diameter. In one example, the food waste is provided at a ratio of ½ fruits and ½ vegetables, with no more than 30 percent of the overall food waste mix being citrus based. The mulched food waste is drained so that when the mulched food is later mixed with the filler to create a compost pre-mixture, the compost pre-mixture has a moisture content of no more than about 30 percent. The mulched food waste is mixed with the filler (e.g., untreated sawdust shavings) to create the compost pre-mixture. The amount of filler that is added is adjusted to provide a carbon to nitrogen ratio in the compost pre-mixture of between about 28:1 to about 39:1. Providing the compost pre-mixture within this range of carbon to nitrogen ratio provides for automatic heating up of the compost pre-mixture to a level that accelerates decomposing of the compost pre-mixture. The compost pre-mixture is aerated frequently to maintain oxygen levels that are needed for the accelerated decomposing. In one example, the compost pre-mixture is aerated every 2 to 4 days, and more preferably about 3 days. The frequent aerating also helps reduce undesirable odors from the composting process.

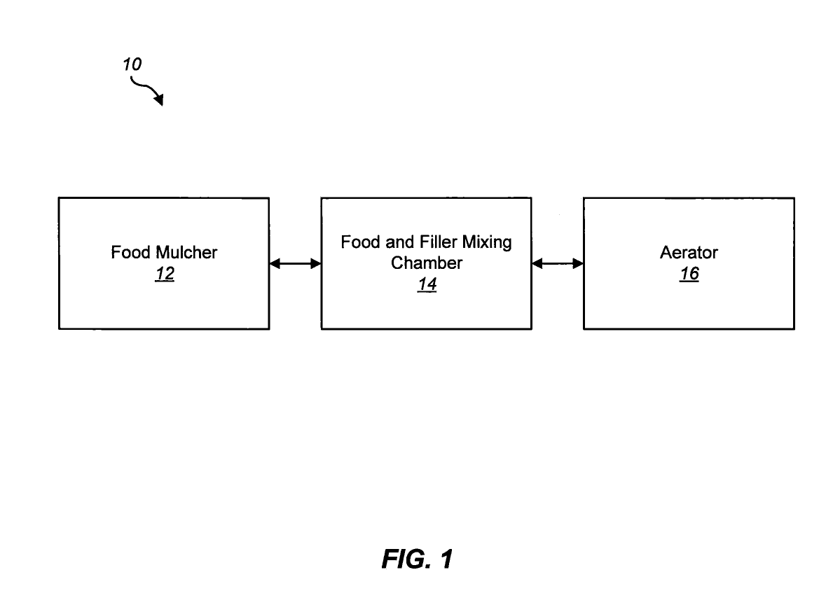

Referring now to FIG. 1, an example system 10 for creating food waste based compost includes a food mulcher 12, a food and filler mixing chamber 14, and an aerator 16. The food mulcher 12 receives fruit, vegetable and other food waste and mulches the food waste to a pre-determined maximum size. The pre-determined maximum size may be in the range of about 0.5 inches to about 2 inches, and more preferably about 1 inch. Typically, the food waste added to food mulcher 12 comprises primarily fruits and vegetables, with a percentage citrus of no greater than 30 percent of the total input of food waste into food mulcher 12. In other examples, one or the other of fruits and vegetables may be added as the food waste to food mulcher 12. Other food waste such as, for example, table scraps from restaurants, expired canned foods, dry foods such as pastas, grains and legumes, or processed foods such as, for example, waste from processing potatoes, corn, and baked goods, including, but in no way limited to breads, cakes, doughnuts, and the like may be included in the food waste add to food mulcher 12. In other examples, the food waste used for system 10 is provided in a condition that does not require mulching and is prepared using other equipment besides food mulcher 12, or can be added directly to the food and filler mixing chamber 14 without further preparation.

The mulched food prepared by food mulcher 12 is typically screened to remove any mulched food that is greater than a pre-determined maximum size. The oversized screened food may be returned to the food mulcher 12 for further processing to a smaller size. Alternatively, the oversized screened food may be disposed of or used for other compost products.

The mulched food provided by food mulcher 12 may also be drained to remove some of the moisture from the mulched food. In one example, the mulched food is stored in containers for a period of time that permits liquids to drain from the mulched food via a draining feature of the container. The mulched food may be stored in piles or in reservoirs or pits where the excess liquid is removed in other ways. In some examples, liquids are removed from the mulched food (e.g., by draining) until a certain average moisture content is reached for a volume of the mulched food.

The drained and screened mulched food is combined with a filler in the food and filler mixing chamber 14. The amount of filler mixed with the mulched food may be adjusted to reach a desired carbon to nitrogen ratio. Typically, the desired carbon to nitrogen ratio is within the range of about 28:1 to about 39:1. This range of carbon to nitrogen ratio has, in at least some examples, provided for automatic heating up to a temperature range that maintains and even accelerates the rate of decomposition, especially when moisture content is within the ranges discussed below. Typically, the temperature range desired for the mixture of food waste and filler (i.e., a compost pre-mixture) is in the range of about 100° F. to about 180° F., and more preferable in the range of about 120° F. to about 160° F. The compost pre-mixture may be tested to confirm the carbon to nitrogen ratio is within the desired range of ratios, with additional filler being added as needed to attain the desired ratio.

The compost pre-mixture of the mulched food and filler typically has a moisture content in the range of about 25% to 35%, and more preferably about 30%. The moisture content of the compost pre-mixture may be tested at a laboratory. An alternative on-site and relatively crude way to test the moisture content of the compost pre-mixture is to grab a handful of the compost pre-mixture and apply a squeezing force. If more than one drop of liquid is squeezed out of the handful of compost pre-mixture, then more than about 30% moisture content is present. If no drop of liquid is expressed from the handful of compost pre-mixture, then less than about 30% moisture content is present.

Additional moisture may be added to the compost pre-mixture by adding mulched food that require little or no draining, adding the liquid that has drained from the mulched food prior to adding the mulched food to the food and filler mixing chamber 14, or adding liquid from another source (e.g., water, liquid-based processed food waste, etc.). Preferably, the liquid that is added to increase the moisture content of the compost pre-mixture has a relatively high nutrient content.

The filler used for the compost pre-mixture may comprise sawdust. The sawdust may have a size in the range of about ¾ inch or smaller, and more preferably ⅜ of an inch or smaller. Any type of wood may be used for the sawdust. In one example, ⅔ cubic yard of sawdust is mixed with about 1,000 lbs. of un-mulched waste food to create a compost pre-mixture having a carbon to nitrogen ratio within the ranges disclosed here. This ratio of unmulched food to sawdust may create about one cubic yard of compost having a weight of approximately 500 lbs.

The food and filler mixing chamber 14 may include a mixing feature such as an auger, stirring arm or other device. In other examples, the food and filler mixing chamber 14 is an enclosed container, while in other examples includes an open pit or a pile wherein the mixing and aerating occurs using a vehicle such as a skid steer loader, tractor, blower, or other device that provides a mixing of the mulched food and filler.

The compost pre-mixture may be arranged in windrows. The windrows may have a maximum height and width so that the compost pre-mixture can maintain a desired temperature (e.g., in the range of 120° F. to 160° F.). The size and shape of the windrow may provide for easier aeration by aerator 16 (e.g., moving the windrow using, for example, a snow blower, scraper, auger, or other device). In one example, the aerator 16 is a snow blower attached to a powered vehicle such as a skid steer loader or tractor. The snow blower moves a windrow of the compost pre-mixture to a different position, typically directly adjacent to the windrow to form a new windrow. Many other types of aerators and configurations for storing and moving the compost pre-mixture to provide the desired aeration of the compost pre-mixture are possible.

The aerator 16 typically aerates the compost pre-mixture about every 1 to 10 days, and more preferably about every 2 to 4 days. The aeration not only oxygenates the compost pre-mixture to assist in the composting process, but also helps reduce odors generated from the composting process.

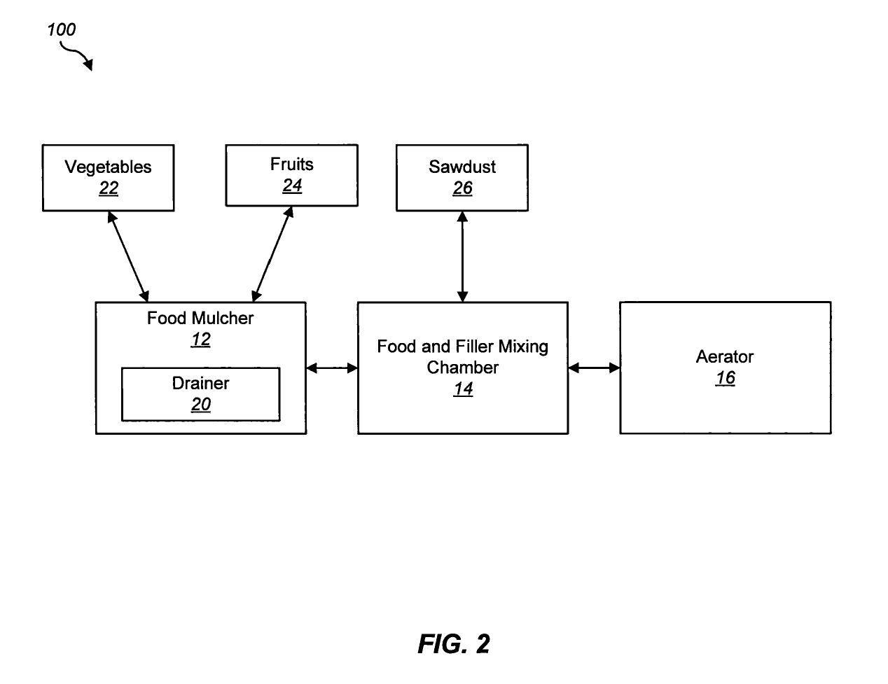

Referring now to FIG. 2, another example system 100 includes a drainer 20 that drains liquid from vegetables 22 and fruits 24 that are mulched by the food mulcher 12. The drainer 20 may be part of the food mulcher 12 or food and filler mixing chamber 14. The drainer 20 may be part of a storage container that holds the mulched food waste before delivery to the food and filler mixing chamber 14.

Sawdust 26 is added as a filler to the food and filler mixing chamber 14 to mix with the mulched food that has been drained by drainer 20. Although not shown, a screener or other size control feature may be included with the food mulcher 12 or at some point between the food mulcher 12 and food and filler mixing chamber 14 to remove mulched food having a size greater than a pre-determined maximum size. The sawdust 26 may also be screened to remove any sawdust having a size greater than a pre-determined maximum size.

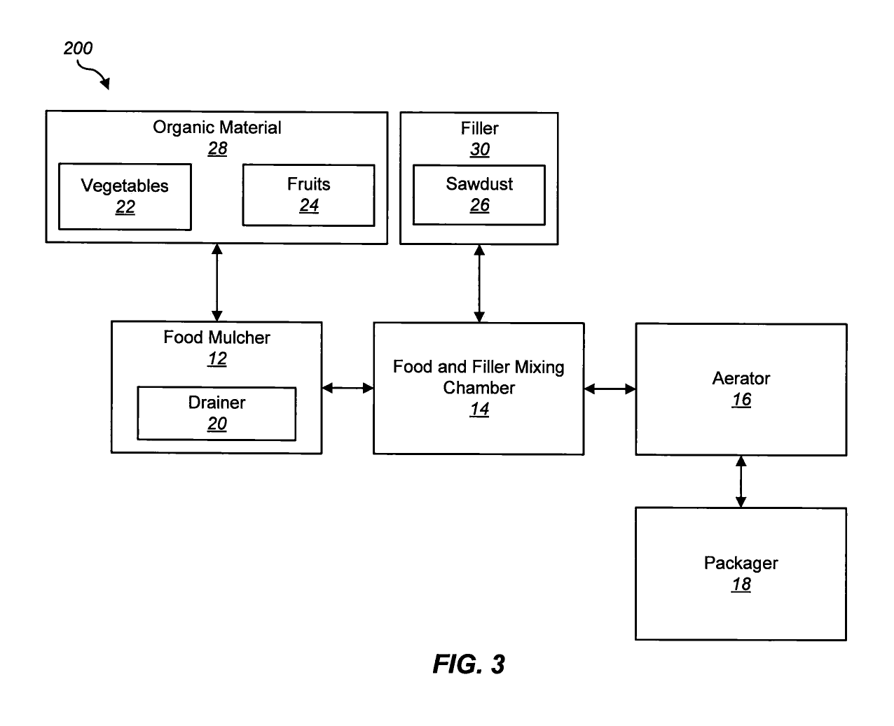

Referring to FIG. 3, another example system 200 provides input of organic material 28 to the food mulcher 12. The organic material 28 may include the vegetables 22 and fruits 24. The organic material 28 may include other organic material that is typically food waste as defined above. There may be some types of organic materials that are usually not edible but are high in nutrient content that may be desirable for use in system 200. Typically, the organic material 28 does not include animal-based product such as manure, or green waste such as leaves, grass clippings and other yard waste.

System 200 also includes filler 30 that may be added with the mulched food at the food and filler mixing chamber 14. The filler 30 may include the sawdust 26 or other filler material. The filler 30 may be used to control the carbon to nitrogen ratio of the compost pre-mixture generated at the food and filler mixing chamber 14. Other types of filler 30 besides sawdust 26 may be used including, for example, hay, shredded paper, pine needles, straw, peanut shells, rice hulls, leaves, and the like.

System 200 may also include a packager 18 that is used to package the compost generated from the compost pre-mixture that is aerated by aerator 16. The packager 18 may operate to package the compost into containers or packaging that can be distributed to retailers.

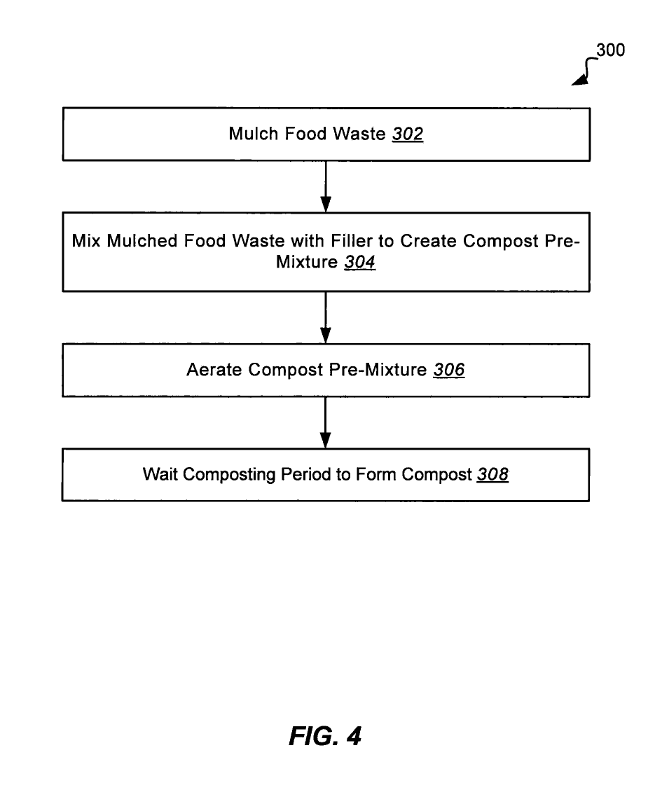

Referring now to FIG. 4, an example method 300 of creating food-based compost includes a first step 302 of mulching food waste. The mulched food waste is mixed with filler to create a compost pre-mixture in a step 304. The compost pre-mixture may be void of any animal-based products such as, for example, manure. The compost pre-mixture is aerated in a step 306. A step 308 includes waiting a composting period to form compost from the compost pre-mixture. The composting period may be in the range of, for example, less than one month, and more preferably in the range of about 2 to about 4 weeks.

The food waste mulched in step 302 may include fruits and vegetables and may be limited solely to fruits and vegetables. The food waste may include only one of fruits or vegetables. The food waste may have a percentage citrus content of no greater than 30%. The food waste may include other edible organic material. An additional step may include screening the mulched food waste to remove any mulched food waste having a size greater than a pre-determined maximum size such as, for example, 1 inch in diameter.

The food waste may have a percentage of fruits of about 40% to 60% and a percentage of vegetables of about 40% to 60% in a food waste mixture. The fruits and vegetables included in the food waste may include a variety of fruits and a variety of vegetables, or may include only one type of fruit or one type of vegetable. The types of fruits and vegetables selected for use may influence, among other parameters, a nutrient content of the resultant compost.

The mixing step 304 may include combining the mulched food waste with a filler such as sawdust. The ratio of mulched food waste with filler may be varied to provide a carbon to nitrogen ratio for the compost pre-mixture in the range of about 28:1 to about 39:1. The mulched food waste may have a moisture content that is adjusted to provide for a moisture content of the compost pre-mixture in a range of about 20% to about 40%, and more preferably about 25% to about 35%, with about 30% being most preferred. A moisture content in this range may assist in the automatic raising of a temperature of the compost pre-mixture to a level that accelerates the composting process. The mulched food waste may be drained prior to being mixed with the filler to create the compost pre-mixture in order to obtain the desired moisture content for the compost pre-mixture. The filler may be filtered or screened to remove any filler having a size greater than a pre-determined maximum size (e.g., no greater than about ½ inch or no greater than about ⅜ inch).

Aerating the compost pre-mixture in step 306 may include piling the compost pre-mixture in windrows and aerating the compost pre-mixture with a snow blower or other moving device that moves the windrow from one location to another while mixing and aerating the compost pre-mixture. Aerating the compost pre-mixture increases an oxygen content of the compost pre-mixture. Increased oxygen content may accelerate the composting process for the compost pre-mixture. The compost pre-mixture may include various bacteria that require oxygen as part of the composting process.

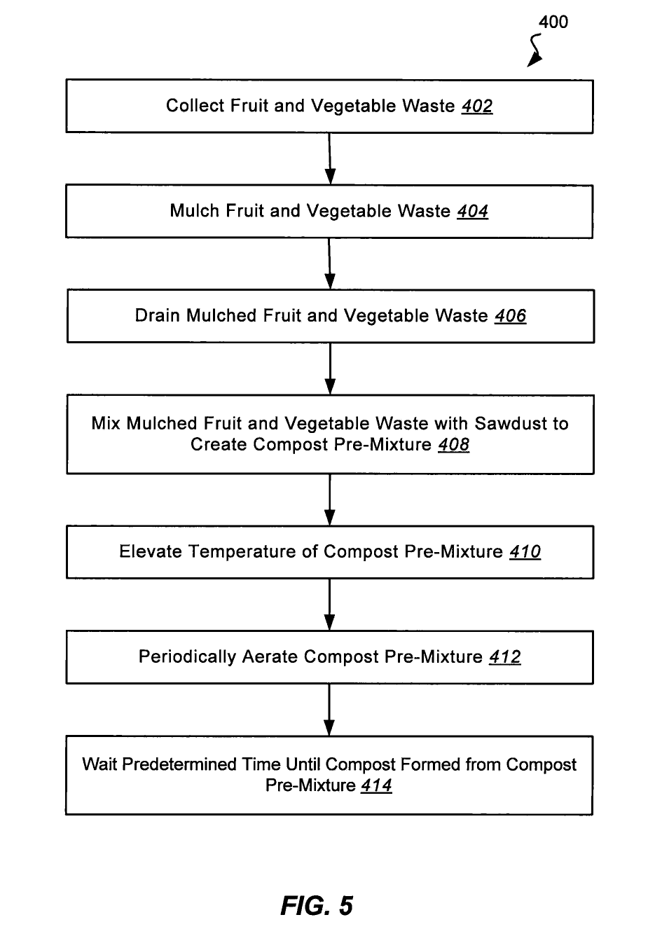

Referring now to FIG. 5, another example method 400 includes a first method step 402 of collecting food waste that includes fruits and vegetables (also referred to as fruit and vegetable waste). The fruit and vegetable waste is mulched in a step 404. The mulched fruit and vegetable waste is drained in a step 406. A step 408 includes mixing the mulched fruit and vegetable waste with sawdust to create a compost pre-mixture. A temperature of the compost pre-mixture is elevated in a step 410. A step 412 includes periodically aerating the compost pre-mixture. A step 412 includes waiting a pre-determined time until compost is formed from the compost pre-mixture.

Collecting the fruit and vegetable waste 402 may include picking up the fruit and vegetable waste from retail sites such as grocery stores, food distributors and food wholesalers, farms, processing plants, restaurants, and food markets. The collecting step 402 may include collecting other food waste besides fruit and vegetable waste, and other organic material including edible organic material and material high in nutrient content. Typically, the food waste used in method 400 does not include animal waste (e.g., manure) or other animal-based product such as meat and bone. The food waste typically does not include packaging (e.g., cardboard), which, while potentially edible, is not usually high in nutrient content.

The method 400 may also include screening or sorting the mulched fruit and vegetable waste to remove any objects that are greater in size than a pre-determined maximum size. The draining of step 406 may include draining for a pre-determined amount of time, draining until a pre-determined amount of liquid is collected, or draining until a rate of flow of the liquid is reduced to a predetermined minimum rate.

The mixing of step 408 may include mixing with other fillers besides sawdust or in addition to sawdust. The mixing may be accomplished using any desired mixing device or process. For example, the mixing may occur within an enclosed container using, for example, an auger or stirring arm. Alternatively, the mixing may occur in an open area using, for example, a vehicle such as a loader, scraper or mobile auger or blower.

The temperature elevation of step 410 may include automatically raising the temperature of the compost pre-mixture to the range of about 120° F. to about 160° F. The automatic temperature elevation may occur at least in part due to the ratio of carbon to nitrogen in the compost pre-mixture. This ratio of carbon to nitrogen may be controlled using an amount of sawdust or other filler that is added in a mixing step 408. The temperature may also be controlled at least in part by the frequency of aerating the compost pre-mixture in a step 412. The temperature may be controlled at least in part by an initial moisture content of the compost pre-mixture. The moisture content may be controlled at least in part by the amount of liquid that is drained in the draining step 406, or the type and condition of the fruit and vegetable waste or other food waste that is mixed in the mixing step 408.

The frequency of aerating in the step 412 is typically in the range of about 1 to 10 days, and more preferably in the range of about 2 to about 4 days, with 3 days being the most preferred frequency of aeration. The time period between aerations may vary when getting closer to the end of the pre-determined time waited in the step 412. For example, the frequency of aeration may change from every 6 days to every 3 days to every day over the course of the pre-determined time.

The aeration in step 412 may be accomplished using, for example, a snow blower attachment on a vehicle such as a skid steer loader or tractor. The snow blower both aerates the compost pre-mixture and moves the compost pre-mixture to a different location (e.g., to form a different windrow adjacent to the original windrow). Other types of aeration devices and processes are possible for the periodic aeration of step 412.

The pre-determined time is typically in the range of about 1 to about 8 weeks, and more preferably in the range of about 2 to 4 weeks, with 3 weeks being most preferred. Providing a carbon to nitrogen ratio in the ranges described above for the compost pre-mixture typically results in a pre-determined time to complete composting in the range of about 2 to 4 weeks. Other parameters such as the frequent aeration and initial moisture content of the compost pre-mixture, alone or in combination with each other and the carbon to nitrogen ratios disclosed herein, may also contribute to the reduced time to complete composting in step 412.

The method 400 may also include a further step of packaging the compost and delivering the compost for sale. The packaged compost may be delivered to retail sites such as home improvement stores and gardening centers. Alternatively, the compost may be delivered in bulk using dump trucks or trailers in an unpackaged form.

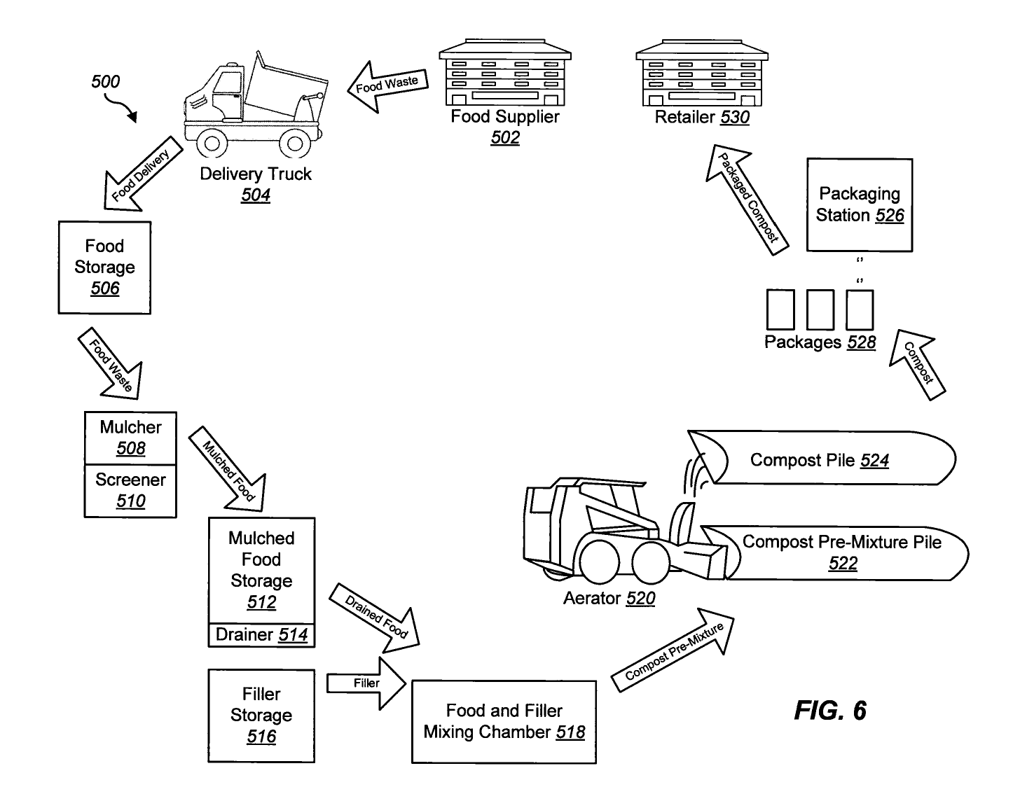

Referring now to FIG. 6, an example system 500 is shown for use in creating food-based compost. A food supplier 502 provides food waste that is delivered by a delivery truck 504 or other delivery means to a food storage 506. The food waste held in food storage 506 is fed to a mulcher 508. The mulcher 508 may include or be associated with a screener 510. The screener 510 may operate to remove mulched food having a size greater than a pre-determined maximum size before the mulched food is delivered to the mulched food storage 512. The mulched food may drain via drainer 514 to remove excess liquid before delivering the mulched food to the food and filler mixing chamber 518.

Filler from filler storage 516 may also be added to the food and filler mixing chamber 518 where the mulched food and filler are mixed together to form a compost pre-mixture. The amount of draining of the mulched food held in mulched food storage 512 influences the moisture content of the compost pre-mixture. The amount of filler added influences a carbon to nitrogen ratio for the compost pre-mixture so that the carbon to nitrogen ratio can be adjusted to be in the range of about 28:1 to 39:1.

The compost pre-mixture is aerated with an aerator 520. The aerator 520 may aerate a compost pre-mixture pile 522. Aerating with the aerator 520 may also move the compost pre-mixture pile 522 physically to a different location to create, for example, a compost pile 524. The compost pile 524 may represent the completion of the composting process wherein the compost pre-mixture has decomposed into compost. A pre-determined amount of time may be required to form the compost from when the compost pre-mixture is originally formed. In one example, the pre-determined period is in the range of about 2 to 4 weeks, and more preferably about 3 weeks. Aeration with the aerator 520 may occur periodically such as, for example, in the range of about every 2 days to about 4 days, and more preferably about every 3 days.

The compost may be delivered to a packaging station 526 where a plurality of packages 528 of compost are formed. The packaged compost may be delivered to a retailer or other distribution site 530. In one example, the packaged compost is delivered to a retailer from which the food waste was originally received.

### Example 1

The following table shows various compost parameters for a sample food-based compost prepared in accordance with the above disclosure. The compost was formed using food waste having 50% vegetables and 50% fruit by weight, which was mixed with sawdust to form a compost pre-mixture having a carbon to nitrogen content in the range of about 28:1 to about 39:1. The following parameters are merely exemplary of one exemplary compost combination according to the present teachings.

| Compost Parameters | Reported as | Test Results |
|---|---|---|
| (Plant Nutrients) | (units of measure) | (wet weight basis) |
| Nitrogen Total N | (TN or TKN + N03-N) | 0.466 |
| Phosphorus | P205 | 0.147 |
| Potassium | K20 | 0.912 |
| Calcium | Ca | 0.297 |
| Magnesium | Mg | 0.490 |
| Moisture Content | %, wet weight basis | 52.03 |
| Organic Matter Content | %, dry weight basis | 68.60 |
| pH | Unitless | 6.90 |
| Soluble Salts | dS/m (mmhos/cm) | 1.33 |
| (electrical conductivity) | | |
| Particle Size or Sieve | Size maximum | 5/8″ |
| | aggregate size, in. | |

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.”

What is claimed is:

1. A method of making food-based compost, comprising:
mulching food to a predetermined maximum size;
mixing mulched food with a filler to create a compost pre-mixture;
aerating the compost pre-mixture;
waiting a composting period until compost is formed from the compost pre-mixture;
wherein the compost does not include animal-based products.

2. The method of claim 1, wherein the food includes fruits and vegetables.

3. The method of claim 1, further comprising screening the mulched food to a maximum of 1 inch.

4. The method of claim 1, further comprising providing the filler at a size of no greater than ⅜ inch.

5. The method of claim 2, wherein the fruits and vegetables are provided at a ratio of 50 percent fruits and 50 percent vegetables by weight.

6. The method of claim 1, wherein the food includes citrus fruit at a ratio of no more than 30 percent by weight.

7. The method of claim 1, wherein a moisture content of the compost pre-mixture is no more than 30 percent.

8. The method of claim 1, wherein a carbon to nitrogen ratio for the compost pre-mixture is in the range of about 28:1 to 39:1.

9. The method of claim 1, wherein the filler includes sawdust.

10. The method of claim 9, wherein the compost pre-mixture includes a ratio of 1000 lbs. of mulched food for every ⅔ cubic yard of filler.

11. The method of claim 1, wherein the composting period is about 3 weeks.

12. The method of claim 1, wherein aerating occurs every 2 to 4 days.

13. The method of claim 1, wherein the compost does not include dirt.

14. A method of making food-based compost, comprising:
mulching a plurality of fruits and vegetables;
draining the mulched fruits and vegetables;
mixing the mulched fruits and vegetables with sawdust to create a compost pre-mixture having a carbon to nitrogen ratio in the range of about 28:1 to about 39:1;
elevating a temperature of the compost pre-mixture;
periodically aerating the compost pre-mixture;
waiting a predetermined time until a compost is formed from the compost pre-mixture;
wherein the compost does not include animal-based products.

15. The method of claim 14, further comprising providing the plurality of fruits and vegetables at a ratio of 50/50 by weight.

16. The method of claim 14, further comprising screening the mulched fruits and vegetables to a size of no greater than 1 inch.

17. The method of claim 14, further comprising providing the compost pre-mixture at a moisture content of no greater than 30 percent.

18. The method of claim 14, wherein periodically aerating the compost occurs at intervals of every 3 days or less.

19. The method of claim 14, wherein the predetermined time is no longer than 3 weeks.

20. The method of claim 14, wherein the compost does not include dirt.

21. The method of claim 14, wherein the mulched fruits and vegetables make up more than ½ of the compost pre-mixture by weight.

22. A system for creating food-based compost, comprising:
a food mulcher configured to mulch food-based product;
a screener configured to remove mulched food-based product greater than a predetermined size;
a drainer configured to drain liquid from the mulched food-based product;
a food and filler mixing chamber configured to mix the mulched food-based product with a filler to form a compost pre-mixture;
an aerator configured to periodically aerate the compost pre-mixture until the compost pre-mixture changes to compost.

23. The system of claim 22, wherein the food-based product comprises a mixture of fruits and vegetables.

24. The system of claim 22, wherein the filler comprises sawdust.

25. The system of claim 22, wherein the aerator comprises a snow blower.
